(19) United States
(12) Patent Application Publication
(10) Pub. No.: US 2019/0249166 A1
(43) Pub. Date: Aug. 15, 2019

(54) Antidote for Chondroitinase

(71) Applicant: Bing Lou WONG, Irvine, CA (US)

(72) Inventor: Bing Lou WONG, Irvine, CA (US)

(21) Appl. No.: 16/254,739

(22) Filed: Jan. 23, 2019

Related U.S. Application Data
(60) Provisional application No. 62/630,810, filed on Feb. 14, 2018.

(51) Int. Cl. C12N 9/99

(52) U.S. Cl. CPC C12N 9/99

(57) ABSTRACT

The present invention provides a method for inhibiting chondroitinase activity in a substrate by introducing a non-substrate glycosaminoglycan (GAG) to the substrate containing the chondroitinase. The non-substrate GAG is undegradable by chondroitinase. The GAG may be administered as a single dose or in multiple doses. The chondroitinase may be Chondroitinase B, Chondroitinase C, Chondroitinase AC, or Chondroitinase ABC. The substrate may be one or more of dermatan sulfate, hyaluronic acid, chondroitin sulfate, or derivatives thereof. The non-substrate GAG may be from naturally unbranched homo-polysaccharide, unnaturally branched GAG, or a hybrid GAG molecule fused of two or three GAG chains, being produced by chemical synthesis or enzymatic reaction. The non-substrate GAG may be heparin, heparan sulfate, and keratan sulfate. The non-substrate GAG may bind to the active residue of the chondroitinase when being introduced to the substrate such that the chondroitinase is no longer enzymatically active.

### CROSS-REFERENCE TO RELATED APPLICATION

The present application claims priority from the U.S. provisional patent application Ser. No. 62/630,810 filed Feb. 14, 2018, the disclosure of which is incorporated herein by reference in its entirety.

### TECHNICAL FIELD

The present invention relates to a use of a non-substrate glycosaminoglycan (GAG)-based antidote for inhibiting chondroitinase activity in a substrate.

### BACKGROUND

Glycosaminoglycan lyase (GAG lyase), which is a group of enzymes effectively degrading glycosaminoglycan (GAG), have been identified from viruses and a wide range of bacteria. In these micro-organisms, the enzymes are known to be used as a virulence factor for infection or as a digestive enzyme for utilizing GAG as carbon source. The substrates of the enzyme, GAGs, are negatively charged polysaccharides and are the major composition of extracellular matrix (ECM) synthesized in animals. Based on the chemical structure, GAG can be classified into 6 groups: chondroitin sulfate, dermatan sulfate, keratan sulfate, heparin, heparan sulfate, and hyaluronic acid (FIG. 1). Although GAGs share similar structural characteristics, the GAG lyases usually can only target a specific type of GAG and therefore these enzymes are grouped based on their substrate specificity, namely chondroitinase, keratanase, heparinase/heparitinase, and hyaluronidase. Unlike another GAG degrading enzymes (GAG hydrolyases), these GAG lyases degrade GAG through eliminative cleavage, cleaving the hexoamine-uronic acid linkage to form unsaturated oligosaccharide/disaccharide molecules and forming C—C double bonds between C4 and C5 carbon on the uronic acid moiety.

Among these GAG lyases, chondroitinase is the most-studied enzyme, not only because of the important role in bacterial survival and pathogen's virulence, but the high pharmaceutical value of the enzyme. Chondroitinase can be further divided into 4 subtypes as listed in table 1.1 based on the subclass of chondroitin sulfate and other substrates of these enzymes.

TABLE 1.1

| Chondroitinase | Substrate Specificity |
|---|---|
| Chondroitinase B | Dermatan sulfate (CSB*) |
| | Hyaluronic acid (HA). |
| | *Previously named as chondroitin sulfate B |
| Chondroitinase C | Chondroitin sulfate C (CSC) |
| | Hyaluronic acid (HA). |
| Chondroitinase AC | Chondroitin sulfate A (CSA), |
| | Chondroitin sulfate C (CSC) |
| | Hyaluronic acid (HA). |
| Chondroitinase ABC | Chondroitin sulfate A (CSA), |
| | Dermatan sulfate (CSB) |
| | Chondroitin sulfate C (CSC), |
| | Hyaluronic acid (HA). |

The pharmaceutical value of these enzymes comes from the high catalytic digestion capability on chondroitin sulfate, which is the major GAG in biological systems, serving as a structural element of the extracellular matrix, or signaling molecules that control, modify, regulate, and inhibit large amounts of physiological activities. Chondroitin sulfate in the extracellular environment are covalently linked to a wide range of protein, and the combined macromolecules are named as chondroitin sulfate proteoglycan (CSPG), including Aggrecan, Versican, Neurocan, Tenascin, etc.

Among those CSPGs, Aggrecan is the most dominant one which is a large biomolecule composed of three globular structural domains (G1, G2, and G3 domains) at the N-terminal and C-terminal end, with a large extended domain (CS domain) heavily modified with chondroitin sulfate and keratan sulfate. This CSPG is a critical structural element in cartilaginous tissue, and the loss of function of this molecule will result in spondyloepiphyseal dysplasia and premature osteoarthritis. Aggrecan is also dominantly found in the nucleus pulposus (NP) of an intervertebral disc (IVD), providing hydrostatic pressure against compressive loading from the vertebra.

In a normal IVD, the high-water content in nucleus pulposus (NP) gives rise to the hydrostatic pressure supporting the compressive loads arising from body weight and muscle tension. But with increasing age, the nucleus becomes more fibrotic and less gel-like. This is caused by gradually degradation of the chondroitin sulfate chain in the aggrecan molecule, resulting in loss of hydration and function of the disc. In the late stage of the degeneration, significant symptoms, e.g., low back pain and sciatica, may appear and are considered as disc degenerative disease. Another CSPG-related IVD disease is disc herniation: CSPG-containing NP escapes from the center of an IVD through the ruptured annulus fibrosus, the outer structure of the IVD. The escaped NP together with the ruptured annulus fibrosus will compress the nearby nerve root, causing low back pain and sciatica.

Chondroitinases have been proven to be useful for treating this disease by injecting into the NP of the herniated disc to digest the chondroitin sulfate chain of the Aggrecan, reducing the degree of hydration and diminishing the hernia size, and finally decompression of the nerve root. In such a case, chondroitinase is classified as a chemonucleolysis agent, a reagent for removing the nucleus in an intervertebral disc.

Another attractive medical application is the promotion of nerve regeneration. Neurocan, another important CSPG, will be produced in the glial scar by astrocytes under the normal response to a central nervous system (CNS) injury. As CSPG is known to be a strong inhibitor of axon growth, the nerve at the site of injury is normally incapable to be regrowth. Applying chondroitinase in the site of injury to digest the chondroitin sulfate can therefore remove the grow inhibition factor and promote the nerve regeneration.

Other applications of chondroitinase include treatment of ocular diseases.

In the mentioned medical uses of chondroitinase, the substrate, chondroitin sulfate, in the affected area is digested by injection of the enzyme to achieve the treatment effect, and the process is controlled by the dose of injection. However, it is desirable that the action of chondroitinase be more precisely controlled in such a way that the enzyme can function for a defined duration, followed by stopping the enzymatic activity completely at a predetermined time point. This will be particularly beneficial for treatment procedures especially when over-digestion of chondroitin sulfate is detrimental. For example, when using chondroitinase as chemonucleolysis agent for digestion of the nucleus pulposus, accidental over-dosing of the enzyme could happen during the operation or, alternatively, a patient could be overly-sensitive to the effects of chondroitinase. In such a case, the nucleus pulposus will be over digested and the intervertebral disc will lose function and finally result in disc degeneration. To this end, applying a specific inhibitor, or an antidote, can effectively resolve this problem.

To achieve the said “precise control of chondroitinase,” the precise termination of the enzyme reaction is required. This can be done by applying a specific inhibitor to the area under chondroitinase treatment. However, this is particularly difficult for the case of chondroitinase, because the enzyme is highly active and the concentration in the area under treatment is very low. Therefore, the inhibitor for chondroitinase must be highly potent and specific to the enzyme. It has been previously described that chondroitinase can be inhibited by several types of substances, including transition metal ions, fatty acids, and non-substrate GAG. Although the inhibition by transition metal ions is effective, it is not suitable as a therapeutic antidote because metal ions have a small molecular weight and the concentration will be reduced through diffusion into the circulatory system. More importantly, many transition metal ions are toxic to humans. Fatty acids are also readily absorbed into the cytosol by the cell of the tissue under chondroitinase treatment, reducing the concentration for enzyme inhibition in the extra-cellular area. Being large polysaccharides, GAGs are relatively stable in the extra-cellular environment without significant cellular influx or clearance through blood vessel. Indeed, GAGs are the major component of extra-cellular matrix, therefore, they have a very high biocompatibility. As a result, these particular types of molecules are selected for the development of an antidote for chondroitinase.

### SUMMARY OF THE INVENTION

The present invention provides a method for inhibiting chondroitinase activity in a substrate comprising introducing a non-substrate glycosaminoglycans (GAG) to the substrate containing the chondroitinase, wherein said non-substrate GAG is undegradable by the chondroitinase. The non-substrate glycosaminoglycans may be administered as a single dose or in multiple doses over a period of time. The chondroitinase may be Chondroitinase B, Chondroitinase C, Chondroitinase AC, and Chondroitinase ABC. The substrate may be one or more of dermatan sulfate, hyaluronic acid, chondroitin sulfate, or derivatives thereof. The non-substrate GAG may be from naturally unbranched homo-polysaccharide, unnaturally branched GAG, or a hybrid GAG molecule fused of two or three GAG chains, being produced by chemical synthesis or enzymatic reaction. The non-substrate GAG may be heparin, heparan sulfate, and keratan sulfate. The non-substrate GAG may bind to the active residue of the chondroitinase when being introduced to the substrate containing the chondroitinase such that the chondroitinase is no longer enzymatically active to the substrate. The chondroitinase may originate from Proteus vulgaris, being produced recombinantly, or it may be wild-type chondroitinase. Other recombinantly-produced chondroitinases may also be used. The derivatives may comprise chondroitin sulfate proteoglycan. The chondroitin sulfate proteoglycan may be Aggrecan, Versican, Neurocan, or Tenascin.

In another aspect, the present invention provides a method for inhibiting chemonucleolysis in a tissue comprising introducing a non-substrate glycosaminoglycans (GAG) to the tissue being pre-conditioned with a chemonucleolysis agent, wherein said non-substrate GAG is undegradable by the chemonucleolysis agent. The chemonucleolysis agent may be chondroitinase comprising Chondroitinase B, Chondroitinase C, Chondroitinase AC, or Chondroitinase ABC. The method of claim 10, wherein said tissue is rich in a substrate comprising dermatan sulfate, hyaluronic acid, chondroitin sulfate, and the derivatives thereof. The non-substrate GAG may be selected from naturally unbranched homo-polysaccharide, unnaturally branched GAG, or a hybrid GAG molecule fused of two or three GAG chains, being produced by chemical synthesis or enzymatic reaction. The non-substrate GAG may be heparin, heparan sulfate, and keratan sulfate.

The non-substrate GAG may bind to the active residue of the chemonucleolysis agent when being introduced to the tissue pre-conditioned with the chemonucleolysis agent such that the chemonucleolysis agent is no longer enzymatically active to the substrate. The chondroitinase may be recombinantly-formed chondroitinase, and Proteus vulgaris may be used in the manufacture of the chondroitinase or the chondroitinase may be naturally-occurring chondroitinase. The tissue may be nucleus pulposus of intervertebral disc from a vertebra may be ocular tissue or any other tissue that may be degraded by chondroitinase. The derivatives may comprise chondroitin sulfate proteoglycan. The chondroitin sulfate proteoglycan may comprise Aggrecan, Versican, Neurocan, and Tenascin.

### BRIEF DESCRIPTION OF DRAWINGS

FIG. 1: Structure of glycosaminoglycans

FIG. 2: Model of heparin binding to chondroitinase ABC. FIG. 2a, the interaction between the key active residues of chondroitinase ABC (HIS 501, TYR508, and ARG 560) and heparin; FIG. 2b, the interaction between the residues in the active pocket of chondroitinase ABC (ASN 656, LYS 567, and ASP 658) and heparin.

FIG. 3: Activity of chondroitinase ABC. Plot (1) change in absorbance against time of solution contain 0.01 U/ml chondroitinase ABC and 0.1 mg/ml chondroitin sulfate C, (2) change in absorbance against time of solution contain 0.0125 mg/ml heparin, 0.01 U/ml chondroitinase ABC, and 0.1 mg/ml chondroitin sulfate.

FIG. 4: EMSA of Chondroitinase ABC and heparin, gel stained with Coomassie blue chondroitinase ABC.

FIG. 5: EMSA of Chondroitinase ABC and heparin, gel stained with Alcian blue for heparin.

FIG. 6: Time profile of the absorbance at 232 nm of disc dissolution assay.

FIG. 7: Time profile of the inhibition of chondroitinase ABC by heparin.

FIG. 8: Tomographic images of rabbit spine injected with chondroitinase ABC and heparin.

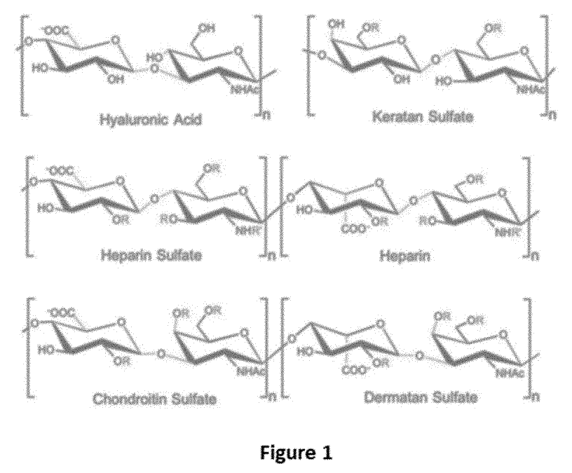

### DETAILED DESCRIPTION OF THE INVENTION

According to the present invention, it was determined that the capability of GAGs used as antidotes for chondroitinase activity would be highest when the substances are non-degradable by chondroitinase; such substances include heparin, heparan sulfate, and keratan sulfate. The GAG polymer may be of various lengths and produced from by different methods and different sources, including cell culture, bacterial fermentation, extraction from animal tissues, synthesis or semi-synthesis from biological-based or chemical-based precursors.

GAGs are naturally unbranched homo-polysaccharides, however, unnaturally branched GAG, a hybrid GAG molecule fused of two or three GAG chains, which could be produced by chemical synthesis or enzymatic reaction, may also be used as antidote as described in this invention when having a high inhibition effect on chondroitinase. A preferred embodiment in the present invention is using heparin as an antidote to chondroitinase activity. Various well-known techniques may be used to produce pharmaceutical grade heparin, examples of which have been described.

The GAG-based antidote described here can be applied to all the sub-types of chondroitinases described in the previous section, which include chondroitinase B, chondroitinase C, chondroitinase AC, and chondroitinase ABC. A preferred embodiment in the present invention is chondroitinase ABC from Proteus vulgaris.

There are few studies on specific inhibition of chondroitinase by GAGs, especially interaction between heparin and chondroitinase ABC. Applying computation modeling and molecular docking to study the interaction between non-substrate GAG and chondroitinase can reveal the mechanism of inhibition and feasibility of blockage of active residues in the enzyme. The modeling can be conducted by commercially available software packages including but not limited to MOE from Chemical Computing Group, Discovery Studio from BIOVIA, ICM-Pro from Molsoft LLC, or general public license software like Autodock, FTDock, etc. In this invention, Autodock is used as an example for demonstrating the molecular interaction between heparin and chondroitinase ABC in silica, which is shown in example 1.

Apart from in-silica evaluation of interaction between GAG and chondroitinase, experimental analysis can also be conducted for obtaining more detail information about the inhibitory effect and antidote capability of GAG. Enzyme inhibition assay which can directly show the effect of GAG on chondroitinase activity is described below.

Freeze-dried chondroitinase is reconstituted in 0.1-10 mg/ml BSA solution, preferably 1 mg/ml. The chondroitinase solution is serially diluted into a final concentration in the range of 0.065-0.35 U/ml, and more preferably 0.260-0.175 U/ml. Chondroitin sulfate C is dissolved in a buffer at pH 7.0-9.0, preferably at pH 8.0±0.1 with Tris-HCl, sodium phosphate, sodium acetate or sodium borate, preferably 10-250 mM, and more preferably 50 mM Tris-HCl as buffer. The final concentration of chondroitin sulfate C is at the range of 0.2-20 mg/ml. Chondroitinase solution and the chondroitin sulfate C solution are pre-incubated in 37° C. and mixed in a ratio of 1:50. The reaction mixture is further incubated for 20 min for allowing the enzymatic reaction proceeding. The absorbance of the reaction mixture is continuously monitored by spectrophotometer and the rate of the reaction is calculated by multiplying the slope of the absorbance-time curve with millimolar extinct coefficient of the reaction product at 232 nm (which is reported to be 5.5.). The rate of the reaction in the absence of GAG is compared with the rate of reaction in the presence of GAG. The effectiveness of the inhibition can be described by the reduction of the rate of the enzymatic reaction. Significant suppression of the enzymatic reaction can be observed in the above assay, especially when heparin is used for inhibiting chondroitinase ABC from Proteus vulgaris, which is described in example 2.

Further experiments can be applied to show the direct interaction, or more precisely binding, between GAG and chondroitinase. One of the experimental methods that can show the direct interaction between non-substrate GAG and chondroitinase (without introducing other molecule, like the substrate) is the electrophoretic mobility shift (EMSA) assay.

The principle of EMSA is that the mobility of biomolecules under electrophoresis is affected by the size and charge of the biomolecule. If two biomolecules bind to from a complex, e.g., chondroitinase and non-substrate GAG, the size and charge of the complex will be significantly different from the either biomolecules. The overall size will increase and the mobility is usually changed in the gel electrophoresis. In other words, a shift in the position of the biomolecule in the gel will be observed if there is binding between the biomolecules. For studying the interaction between chondroitinase and non-substrate GAG, EMSA is conducted as described. 5-30 μg chondroitinase is mixed with (a) 0.5 molar ratio, (b) equal molar, and (c) 2 folds molar excess of GAG, and incubated at room temperature for at least 20 min, and more preferred 30 min. These mixtures are loaded onto native polyacrylamide gel and subjected to electrophoresis for 2.5 hours at 100 V. Two identical gels are prepared but stained with different staining solution (Alcian blue for GAG, and Coomassie blue for chondroitinase).

The above activity assay and binding assay indicate GAG molecule bind and inhibit chondroitinase through the active site binding. However, these experiments only show the interaction between GAG molecule and chondroitinase in a controlled environment. Indeed, in the real situation of using GAG as antidote to neutralize the enzyme activity of chondroitinase, the condition shall be significantly different from the buffer solution, and shall be surrounded by different type of tissue, body fluid, and biomolecules etc. This matrix effect could significantly affect the interaction between the GAG and Chondroitinase resulting in a different inhibition capability.

In the case of using chondroitinase as a chemonucleolysis agent, the site of injection is a gel-like nucleus pulposus which is dense with chondroitin sulfate, collagen, and the other extra-cellular matrix. To evaluate the effect of these matrices on the inhibition, a disc dissolution assay can be conducted. In the experiment, the nucleus pulposus, of which chondroitin sulfate is composed of 65% dry weight, are collected from bovine tails. The nucleus pulposus samples are then put into (1) buffered solution, preferably, but not limited to, phosphate buffered saline with sodium lactate, (2) solution 1 in the presence of chondroitinase, preferably but not limited to chondroitinase ABC, and (3) solution 2 in the presence of non-substrate GAG, preferably, but not limited to heparin. Nucleus pulposus samples are incubated in the solution at room temperature for 10-30 hours, preferably 20 hours. During the incubation, the nucleus pulposus samples slowly decompose in the buffered solution, where in the presence of chondroitinase, specific degradation of chondroitin sulfate can be observed. The result is releasing the enzymatic reaction product, chondroitin sulfate tetrasaccharide and/or disaccharide which strongly absorbs UV light at 232 nm. In such case, the chemonucleolysis process in the intervertebral disc matrix is demonstrated.

Further, the antidote action of non-substrate GAGs can be demonstrated in this experiment. Introducing the non-substrate GAG, e.g. heparin, into the solution together with the chondroitinase, results in suppression of the release of chondroitin sulfate tetrasaccharide and/or disaccharide from the nucleus pulposus sample, is therefore a strong indication of the inhibitory and antidote effect on the enzyme. This inhibition of tetrasaccharide/disaccharide release is reflected by the reduction of the absorbance at 232 nm when compared with the buffer solution in the presence chondroitinase only. The detailed experimental result is shown in example 4.

In one embodiment, the inhibition of chondroitinase ABC by heparin could be at a particular time point during the enzymatic treatment, so that the enzymatic action can be precisely terminated. This can be demonstrated by introducing a suitable concentration of heparin into the reaction matrix, in vitro. One of the examples for demonstrating this is using the chondroitin sulfate harvested from intervertebral disc mixed with chondroitinase ABC. The enzymatic reaction in the mixture, which is the digestion of the chondroitin sulfate polymer into unsaturated oligosaccharide or disaccharide, can be monitored at 232 nm with incubation in 37° C. The reaction can be incubated for 5 hours and at 1.5 and 2.5 hours, with suitable concentrations of heparin including, but not limited to, 0.1-0.01 times the chondroitin sulfate, being introduced for evaluating the time response of the inhibition effect. Detailed experimental results are illustrated in example 5.

Being used as an antidote, the non-substrate GAG of the present invention is effective in vivo. In the case of chemonucleolysis by chondroitinase ABC, the enzyme is injected into the nucleus pulposus for partial digestion. The effective antidote will be able to inhibit the activity of chondroitinase ABC intradiscally, as well as the reduction of the size of the intervertebral disc. To demonstrate this, various levels of chondroitinase ABC are injected into different intervertebral discs of New Zealand rabbits together with heparin. When comparing to the control animal, for which the intervertebral disc is injected with Chondroitinase ABC only, the reduction of the disc height can only be observed in the control animal. The detailed experimental result is illustrated in Example 6.

### EXAMPLE 1

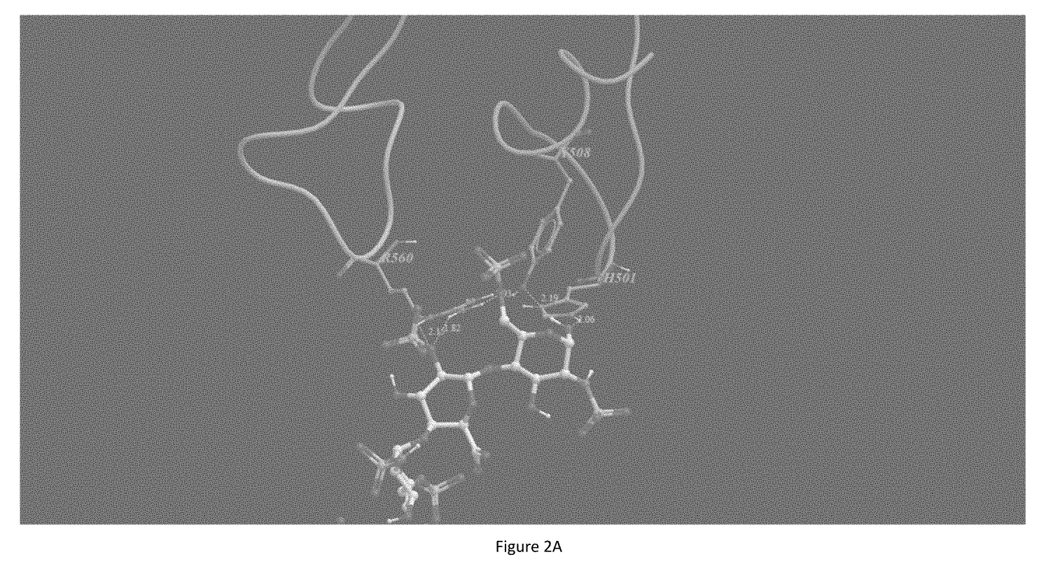

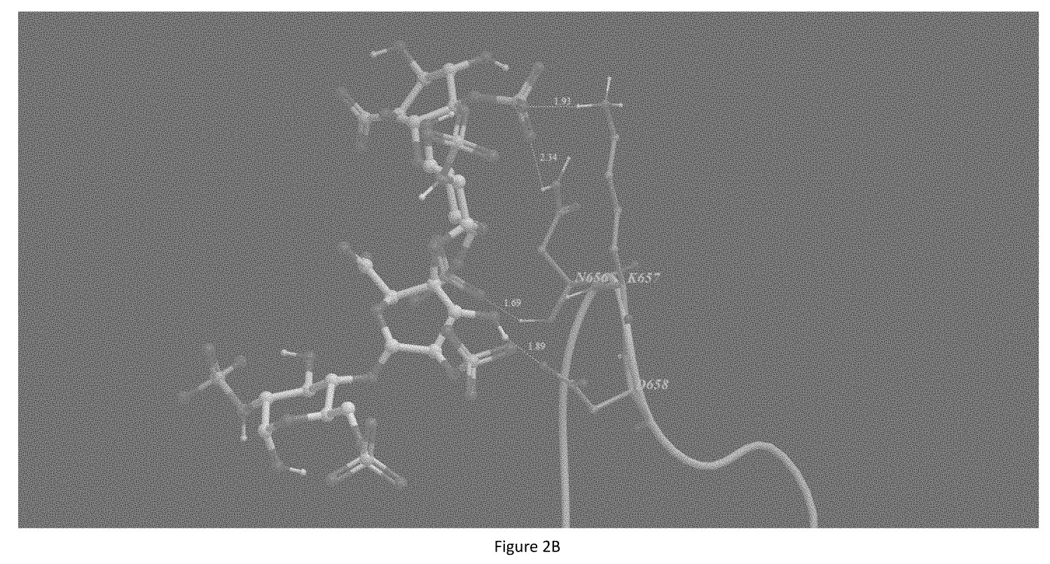

A Chondroitinase ABC model was generated from the crystal structure previously published (Huang W, Lunin V, Li Y, Suzuki S, Sugiura N, Miyazono H, Cygler M, Journal of Molecular Biology, (2003) 328, 623-634). A model of heparin tetrasaccharide was extracted and modified from the crystal structure of heparin (Khan, S, Gor, J, Mulloy, B, Perkins, S J, (2010) Journal of Molecular Biology 395: 504-521). The docking of the heparin tetrasaccharide to the chondroitinase ABC model was performed using Autodock v4.2. The heparin tetrasaccharide was docked within a grid box covering the whole active site with a size of 90×84×78 points using a spacing of 0.375 A. The parameters of the docking consisted of 30 Lamarckian Genetic Algorithm runs using 2,500,000 energy evaluations and a population size of 150 individuals. The docking result showed that the heparin tetrasaccharide had intensive interaction to the active residue of chondroitinase ABC, including His 501, Tyr 508, Arg 560, and His 561. In the model, the heparin tetrasaccharide molecule also has intensive hydrogen bonding network and electrostatic interaction with ASN 656, Lys 657, ASP 658, HIS 712 (FIG. 2a and b). This model showed that the heparin could bind and block the active site of chondroitinase with intensive charge and hydrogen-bond interaction.

### EXAMPLE 2

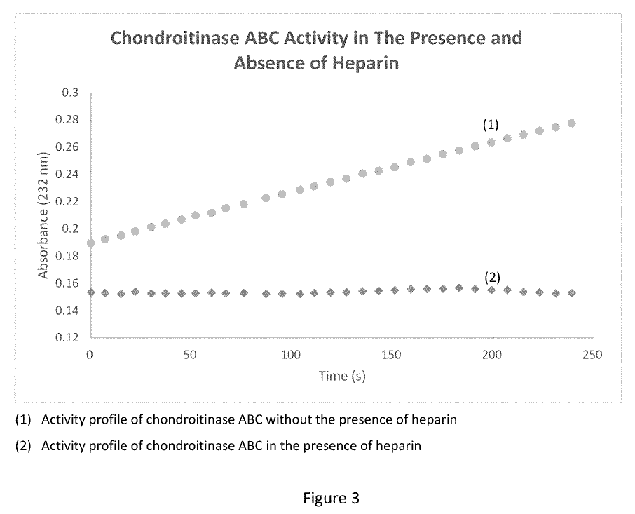

2 mg/ml Chondroitin sulfate C solution was prepared by dissolving in buffer at pH 8.0 Tris-HCl (50 mM). Freeze-dried chondroitinase ABC was reconstituted in 1 mg/ml BSA solution and was serially diluted to 0.5 U/ml by 1 mg/ml BSA. Both solutions were incubated at 37° C. for 5 min separately and were then mixed in a 3.5 ml quartz cuvette. The final concentration of chondroitinase ABC become 0.01 U/ml and 0.1 mg/ml chondroitin sulfate C. The reaction mixture was further incubated at 37° C. for 20 min in spectrophotometer and the absorbance at 232 nm was continuously monitored. The experiment was repeated in the presence of 0.0125 mg/ml heparin. In FIG. 3, the absorbance of the solution without the presence of heparin at 232 nm increased linearly. However, in the presence of 0.0125 mg/ml heparin, the enzymatic reaction of chondroitinase was completely inhibited.

### EXAMPLE 3

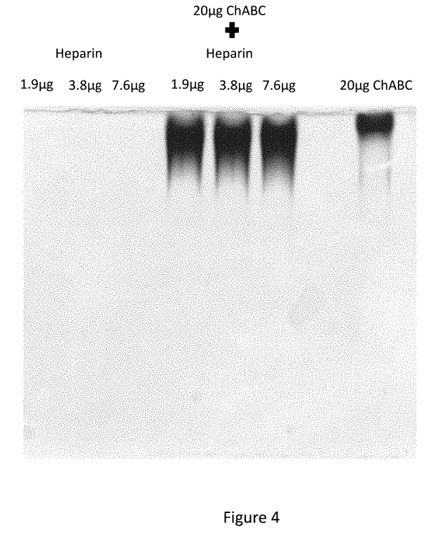

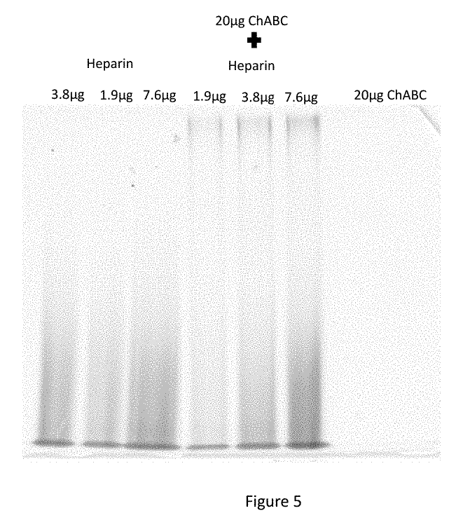

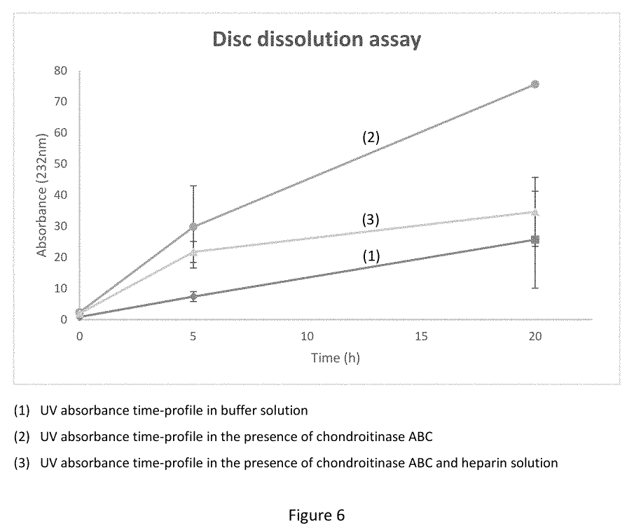

EMSA was conducted by mixing 20 μg chondroitinase ABC (110 kDa, about 0.2 nmol) with 3.8 μg heparin (17-19kDa, about 0.2 nmol) at room temperature for 30 min. Each reaction mixture (16 μl), as well as samples with heparin and chondroitinase ABC, were mixed with 4 μl of 5× native loading buffer. The samples were loaded onto two separate 6% native gels for electrophoresis at 100V for 2.5 hours. After the electrophoresis, one gel was stained with Coomassie blue the other was stained by Alcian Blue. The gels are shown in FIG. 4 and FIG. 5. It can be seen in the gel stained with Alcian Blue (FIG. 4) that heparin is completely moved to the bottom of the gel in the absence of chondroitinase ABC. When Chondroitinase ABC is present, there is a significant amount of GAG present in the band that overlaps with the band of chondroitinase ABC. In other words, heparin is being retained by chondroitinase ABC, indicating that heparin binds tightly to the enzyme.

### EXAMPLE 4

In disc dissolution assay, 6 intervertebral discs were obtained from a bovine tail. The nucleus pulposus were dissected from the intervertebral disc and cut into regular sizes with wet weight in the range of 0.44-0.75 g. The 6 nucleus samples were equally divided into 3 groups and immersed into 5 ml of 3 different solutions, (1) phosphate buffered saline (PBS, pH 7.4) with 10 mM sodium lactate, (2) PBS with 10 mM sodium lactate in the presence of 1U/ml chondroitinase ABC, and (3) PBS with 10 mM sodium lactate in the presence of 1U/ml chondroitinase ABC and 1 mg/ml heparin. The absorbance at 232 nm of each solution was measured at 0 hours, 5 hours, and 20 hours after the immersion of the nucleus pulposus. The increase in absorbance was predominantly due to the dissolution of the small-molecule-containing unsaturated carbon-carbon double bond from the nucleus pulposus. In the presence of chondroitinase ABC, chondroitin sulfate in the nucleus pulposus was rapidly digested into chondroitin sulfate tetrasaccharide or disaccharide with carbon-carbon double bond on the on the uronic acid moiety. As shown in FIG. 5, the absorbance at 232 nm of the solution containing PBS and sodium lactate slowly increased throughout the whole period of the experiment. When compared with the sample containing chondroitinase ABC, the absorbance increased remarkably, which was due to the dissolution of chondroitin sulfate tetrasaccharide or disaccharide. The presence of heparin significantly reduced the absorbance of the solution, which was a strong indication of inhibition of the action of chondroitinase ABC, and the enzymatic digestion of chondroitin sulfate.

### EXAMPLE 5

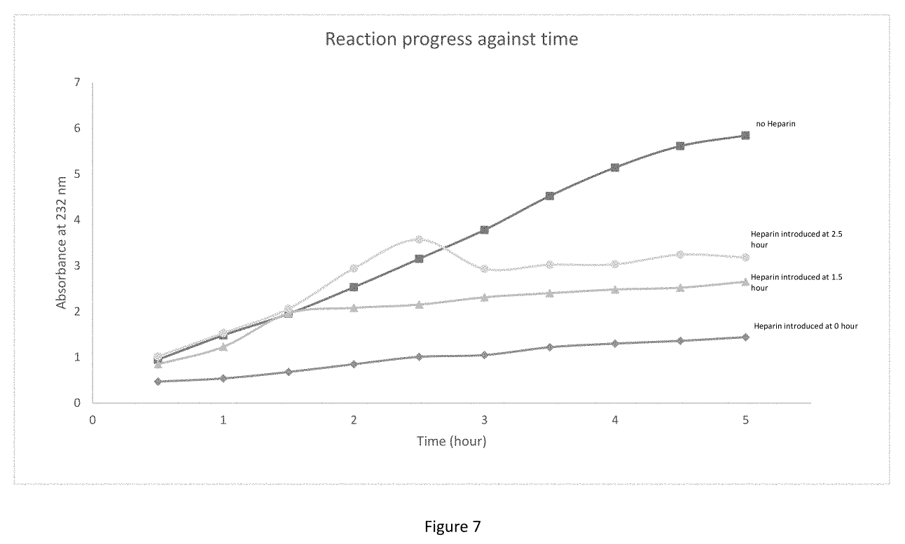

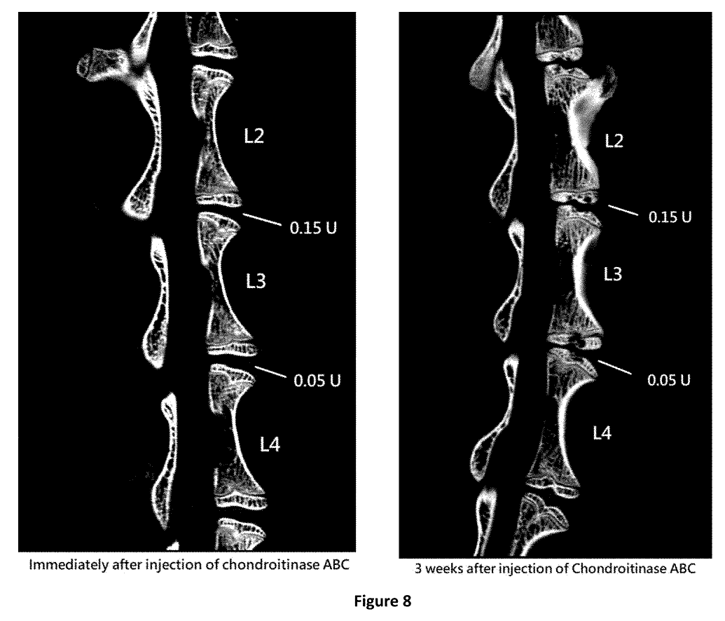

Chondroitin sulfate and chondroitinase ABC were introduced into 50 mM Tris HCl buffered solution (pH 7.6), to a final concentration of 1 mg/ml and 0.001 U/ml. The reaction mixture was aliquoted into 4 equal portions and separately incubated in 37° C. water bath for 5 hours. At time of 0 hour, 1.5 hour, and 2.5 hour, 0.01 mg/ml heparin was introduced into the reaction mixture separately into 3 reaction mixture, where the remaining reaction mixture was sampled and monitored at the same time interval as the 3 reaction mixture with heparin introduction. The progress of the enzymatic reaction was monitored by measuring the absorbance at 232 nm of a sample taken from the reaction mixture every 30 min. The reaction progress represented by the absorbance was plotted against time, shown in FIG. 7. It can be observed that the rate of increase in absorbance of the reaction mixture was significantly reduced and the enzymatic activity remained at a low level when comparing with the reaction mixture without heparin. This also proved that the inhibition of the enzymatic reaction remains stable and constant throughout the reaction period.

### EXAMPLE 6

New Zealand rabbits weighing 2.5-3.0 kg were divided into two groups: (1) injection of Chondroitinase ABC (0.05 U and 0.15 U) and heparin (0.35 mg); and (2) injection of chondroitinase ABC (0.05 U and 0.15 U). Before injection of chondroitinase ABC with or without heparin, rabbits were fixed in the dorsal position and anesthetized by 10% chloral hydrate (1 ml/kg). The intervertebral disc (IVD) was located by means of portable x-ray fluoroscope and the injection of the chondroitinase ABC with or without heparin was under fluoroscopic guidance. Rabbits were sacrificed just after the injection and 3 weeks after of injection for collecting the spine. The disc height index (DHI) analyzed by using LaTheta™ Micro-CT scanners (Hitachi-Aloka) at a resolution of 48 um (FIG. 7) and is listed in Table 2. The IVD injected with 0.5 U and 0.15 U chondroitinase ABC only showed a significantly reduction of DHI, whereas in the group with injection of chondroitinase ABC followed by 0.35 mg heparin, the DHI at 0.05 U chondroitinase was not reduced, i.e., 0.35 mg heparin significantly inhibited the DHI reduction caused by 0.05 U chondroitinase ABC. At a higher concentration of chondroitinase ABC (0.15 U), 0.35 mg heparin also inhibited the DHI reduction but the inhibitory effect was not as significant as at the lower concentration of chondroitinase ABC. This indicated that the heparin could inhibit the activity of chondroitinase ABC in vivo as well as the disc height reduction.

TABLE 2

| Group | | | |
|---|---|---|---|
| | Chondroitinase | DHI | |
| Sampling time | ABC injected | Without heparin | With heparin |
| 0 day after injection | 0.05 U | 0.082 | 0.081 |
| | 0.15 U | 0.071 | 0.085 |
| 3 weeks after injection | 0.05 U | 0.044 | 0.082 |
| | 0.15 U | 0.048 | 0.066 |

While in the foregoing specification the disclosed compounds, compositions, and methods have been described in relation to certain forms thereof, and many details have been put forth for the purpose of illustration, it will be apparent to those skilled in the art that the disclosed compounds, compositions, and methods are susceptible to additional forms and that certain of the details described herein can be varied considerably without departing from the basic principles of the invention.

All references cited herein are incorporated by reference in their entirety. The disclosed compounds, compositions, and methods can be embodied in other specific forms without departing from the spirit or essential attributes thereof and, accordingly, reference should be made to the appended claims, rather than to the foregoing specification, as indicating the scope of the disclosed compounds, compositions, and methods.

1. A method for inhibiting chondroitinase activity in a substrate comprising introducing a non-substrate glycosaminoglycan (GAG) to the substrate containing the chondroitinase, wherein said non-substrate GAG is undegradable by the chondroitinase.

2. The method of claim 1, wherein said chondroitinase is selected from Chondroitinase B, Chondroitinase C, Chondroitinase AC, and Chondroitinase ABC.

3. The method of claim 1, wherein said substrate includes one or more of dermatan sulfate, hyaluronic acid, chondroitin sulfate, or derivatives thereof.

4. The method of claim 1, wherein said non-substrate GAG is selected from naturally unbranched homo-polysaccharide, unnaturally branched GAG, or a hybrid GAG molecule fused of two or three GAG chains, being produced by chemical synthesis, chemoenzymatic synthesis, enzymatic reaction, or production by genetically modified organism.

5. The method of claim 1, wherein said non-substrate GAG comprises heparin, heparan sulfate, and keratan sulfate.

6. The method of claim 1, wherein the non-substrate GAG binds to the active residue of the chondroitinase when being introduced to the substrate containing the chondroitinase such that the chondroitinase is no longer enzymatically active to the substrate.

7. The method of claim 2, wherein said chondroitinase is originated from Proteus vulgaris.

8. The method of claim 3, wherein said derivatives comprise chondroitin sulfate proteoglycan.

9. The method of claim 8, wherein said chondroitin sulfate proteoglycan comprises Aggrecan, Versican, Neurocan, and Tenascin.

10. A method for inhibiting chondroitin degradation by chondroitinase in a tissue, comprising:
accessing a tissue that includes one or more of dermatan sulfate, hyaluronic acid, chondroitin sulfate, or derivatives thereof;
applying a chondroitinase to the tissue, the chondroitinase being selected from one or more of chondroitinase B, chondroitinase C, chondroitinase AC, or chondroitinase ABC, the chondroitinase degrading the tissue;
introducing a non-substrate glycosaminoglycan (GAG) to the tissue degraded by the chondroitinase, the non-substrate glycosaminoglycan being selected from a naturally unbranched homo-polysaccharide, an unnaturally branched GAG, or a hybrid GAG molecule fused of two or three GAG chains, the non-substrate GAG being produced by chemical synthesis, chemoenzymatic synthesis, enzymatic reaction, or production by genetically modified organism, wherein the non-substrate GAG is undegradable by the chondroitinase; and
wherein the non-substrate GAG substantially inhibits the chondroitinase such that the chondroitinase is no longer enzymatically active to the tissue and tissue degradation by the chondroitinase is substantially diminished.

11. (canceled)

12. (canceled)

13. (canceled)

14. The method of claim 10, wherein said non-substrate GAG comprises heparin, heparan sulfate, or keratan sulfate.

15. (canceled)

16. The method of claim 10, wherein said chondroitinase is originated from Proteus vulgaris or is recombinantly-produced chondroitinase.

17. The method of claim 10, wherein said tissue is nucleus pulposus of intervertebral disc from a vertebra.

18. The method of claim 10, wherein said derivatives comprise chondroitin sulfate proteoglycan.

19. The method of claim 18, wherein the chondroitin sulfate proteoglycan comprises Aggrecan, Versican, Neurocan, or Tenascin.
